SEQUENCE LISTING (16 sequences)

SEQ ID NO: 1 - DNA, 21 bp, Artificial Sequence
  Description of Artificial Sequence Synthetic Primer
aaaaggcagagctggagtcgc

SEQ ID NO: 2 - DNA, 20 bp, Artificial Sequence
  Description of Artificial Sequence Synthetic Primer
gtacgaagggctaacaacgg

SEQ ID NO: 3 - DNA, 22 bp, Artificial Sequence
  Description of Artificial Sequence Synthetic Primer
gattcttacgcacgctatttga

SEQ ID NO: 4 - DNA, 20 bp, Artificial Sequence
  Description of Artificial Sequence Synthetic Primer
agcttgctcatgactgggtg

SEQ ID NO: 5 - DNA, 22 bp, Artificial Sequence
  Description of Artificial Sequence Synthetic Primer
ggcagctggaagaagaagagaa

SEQ ID NO: 6 - DNA, 22 bp, Artificial Sequence
  Description of Artificial Sequence Synthetic Primer
gagggctctctgttctcagtga

SEQ ID NO: 7 - DNA, 20 bp, Artificial Sequence
  Description of Artificial Sequence Synthetic Primer
ccccaatgaccctcgagact

SEQ ID NO: 8 - DNA, 19 bp, Artificial Sequence
  Description of Artificial Sequence Synthetic Primer
cctccttccaacccttgcc

SEQ ID NO: 9 - DNA, 20 bp, Artificial Sequence
  Description of Artificial Sequence Synthetic Primer
tctttgaacaccgtgcctcc

SEQ ID NO: 10 - DNA, 20 bp, Artificial Sequence
  Description of Artificial Sequence Synthetic Primer
ctccgcctcgtccagagtat

SEQ ID NO: 11 - DNA, 20 bp, Artificial Sequence
  Description of Artificial Sequence Synthetic Primer
tcaaggcagagaggagggtg

SEQ ID NO: 12 - DNA, 23 bp, Artificial Sequence
  Description of Artificial Sequence Synthetic Primer
gaaagcgaacaaagacattggtt

SEQ ID NO: 13 - DNA, 20 bp, Artificial Sequence
  Description of Artificial Sequence Synthetic Primer
tgctgtggggaagtggttag

SEQ ID NO: 14 - DNA, 23 bp, Artificial Sequence
  Description of Artificial Sequence Synthetic Primer
gaaagcgaacaaagacattggtt

SEQ ID NO: 15 - DNA, 18 bp, Artificial Sequence
  Description of Artificial Sequence Synthetic Primer
tggggcgaagggcaatgg

SEQ ID NO: 16 - DNA, 19 bp, Artificial Sequence
  Description of Artificial Sequence Synthetic Primer
tggagagggggtcagcggt

(19) United States
(12) Patent Application Publication
(10) Pub. No.: US 2007/0087376 A1
(43) Pub. Date: Apr. 19, 2007

(54) Splice variants of pre-mRNA transcripts as biomarkers in idiopathic neurodegenerative diseases

(76) Inventor: Judith Ann Potashkin, Gurnee, IL (US)

Correspondence Address: EVEREST INTELLECTUAL PROPERTY LAW GROUP, P.O. Box 708, Northbrook, IL, 60065, US

(21) Appl. No.: 11/640,724

(22) Filed: Dec. 18, 2006

Related U.S. Application Data
(63) Continuation-in-part of application No. 11/215,842, filed on Aug. 30, 2005.
(60) Provisional application No. 60/605,643, filed on Aug. 30, 2004.

(51) Int. Cl. C12Q 1/68; C07H 21/02

(52) U.S. Cl. 435/6; 536/23.1

(57) ABSTRACT

The present invention discloses a method to discover biomarkers indicative of an idiopathic neurodegenerative disease in a mammalian subject and biomarkers indicative of an idiopathic neurodegenerative disease in the mammalian subject. The biomarker comprises a splice variant mRNA of a precursor-messenger RNA (pre-mRNA) transcript of a gene in the mammalian subject wherein (a) the ratio of the amount of the splice variant mRNA to the amount of another splice variant mRNA of the same precursor-messenger RNA (pre-mRNA) transcript of the same gene is different in the mammalian subject having the neurodegenerative disease as compared to that of a control without the disease; or (b) the ratio of the amount of the splice variant mRNA to the amount of total 18S RNA is different in the mammalian subject having the neurodegenerative disease as compared to that of a control without the disease. The biomarkers can be used to diagnose neurodegenerative diseases in the subject.

### CROSS-REFERENCE TO RELATED APPLICATIONS

This application claims priority from U.S. patent application Ser. No. 11/215,842 filed Aug. 30, 2005, which claims priority of provisional application Ser. No. 60/605,643 filed Aug. 30, 2004, which are both incorporated herein by reference and made a part hereof.

### FEDERALLY SPONSORED RESEARCH OR DEVELOPMENT

This invention was made with government support under United States Army Medical Research and Material Command NETRP grant number W81XWH-05-1-0580. The government has certain rights in the invention.

### TECHNICAL FIELD

The present invention generally relates to biomarkers indicative of idiopathic neurodegenerative diseases, such as Parkinson's disease and Alzheimer's disease, in a mammalian species.

### REFERENCE TO SEQUENCE LISTING

A sequence listing is included as a part of this disclosure and all information contained therein is incorporated herein by reference.

### BACKGROUND OF THE INVENTION

Neurodegenerative diseases can be quite debilitating. Parkinson's disease, for example, is characterized by the progressive and selective loss of nigrostriatal dopamine (DA) neurons. Most cases of Parkinson's disease are sporadic, suggesting a strong environmental influence. The molecular basis of idiopathic Parkinson's disease remains unknown. Alzheimer's disease is also characterized by the progressive loss of brain cells.

In view of the foregoing, it would be beneficial to have a method of diagnosing neurodegenerative disease, such as Parkinson's disease and Alzheimer's disease, as early as possible. In this regard, it would be beneficial to be able to monitor the progression of disease as well as the efficacy of treatment. It is an object of the present invention to provide such methods. This and other objects and advantages of the present invention, as well as additional inventive features, will become apparent from the detailed description provided.

### BRIEF SUMMARY OF THE INVENTION

In an embodiment, the present invention provides a method for discovering biomarkers indicative of an idiopathic neurodegenerative disease in a mammalian species. The method comprises (a) providing a test subject from the mammalian species, the subject having been exposed to an environmental factor that causes the neurodegenerative disease or having been positively diagnosed with the neurodegenerative disease; (b) obtaining RNA from a tissue of the test subject; (c) determining the amounts of a first splice variant mRNA of a precursor-messenger RNA (pre-mRNA) of a gene of the test subject, a second splice variant mRNA of the same pre-mRNA of the same gene and the total RNA; (d) determining for the test subject a first ratio of the amount of first splice variant mRNA to the amount of the second variant mRNA or determining for the test subject a second ratio of the amount of the first splice variant mRNA to the amount of total 18S RNA; (e) obtaining a third ratio of a control subject of the amount of the first splice variant mRNA to the amount of the second splice variant mRNA or a fourth ratio of a control subject of the amount of the first splice variant mRNA to the amount of total 18S RNA; (f) comparing the first ratio to the third ratio to determine a first difference or comparing the second ratio to the fourth ratio to determine a second difference; and (g) identifying the first splice variant mRNA as a biomarker indicative of the neurodegenerative disease in the mammalian species if the first difference or the second difference is not zero, which can be positive or negative. The mammalian species can be any species, including but is not limited to mouse or human. The neurodegenerative disease can be any neurodegenerative disease, such as but is not limited to Parkinson's disease and Alzheimer's disease. The exposure to the environmental factor can be unintentional or intentional, and can be acute or chronic. The tissue to obtain the RNA is preferably blood or cerebral spinal fluid (CSF).

In another embodiment, the present invention discloses a biomarker indicative of an idiopathic neurodegenerative disease in a mammalian subject of a mammalian species, the mammalian subject having a first amount of a first splice variant mRNA of a precursor-messenger RNA (pre-mRNA) transcript of a gene in the mammalian subject, a second amount of a second splice variant mRNA of the same precursor-messenger RNA (pre-mRNA) transcript of the same gene and a total amount of 18S RNA, and the biomarker for the mammalian species comprises the first splice variant mRNA wherein the first splice variant mRNA satisfies one of the conditions selected from the group consisting of: (a) a ratio of the amount of the first splice variant to the amount of the second splice variant of the mammalian subject having been exposed to an environmental factor known to cause the neurodegenerative disease or having been positively diagnosed with the neurodegenerative disease is different from a ratio of the amount of the first splice variant to the amount of the second splice variant in a control subject who does not have the neurodegenerative disease; and (b) a ratio of the amount of the first splice variant to the amount of 18S RNA of the mammalian subject having been exposed to an environmental factor which causes the neurodegenerative disease or having been positively diagnosed with the neurodegenerative disease is different from a ratio of the amount of the first splice variant to the amount of total 18S RNA in a control subject who does not have the neurodegenerative disease. The difference can be positive or negative. The mammalian species can be any species, including but is not limited to mouse or human. The neurodegenerative disease can be any neurodegenerative disease, such as but is not limited to Parkinson's disease and Alzheimer's disease. The exposure to the environmental factor can be unintentional or intentional, and can be acute or chronic. The tissue to obtain the RNA is preferably blood or cerebral spinal fluid (CSF).

Examples of biomarkers indicative of neurodegenerative diseases in the present invention include but are not limited to splice variant mRNAs of pre-mRNAs from the genes FosB, RGS9, Ania6, AChE and NDUFS4. These biomarkers can be used in the diagnosis and prognosis of neurodegenerative diseases. They can also be used to screen for potential therapeutic agents for the treatment of neurodegenerative diseases.

### BRIEF DESCRIPTION OF THE DRAWINGS

FIG. 1 is a schematic diagram indicating the alternative splicing of FosB pre-mRNA to form ΔFosB and FosB mRNA (E1=exon 1, E2=exon 2, E3=exon 3, E4=exon 4, and E5=exon 5);

FIG. 2 shows the levels of FosB splice variants in the blood of Parkinsonian mice described in Example 1. The mice were euthanized 3 days (D) after acute MPTP treatment, or 3 days (D) or 3 weeks (WK) after chronic MPTP administration;

FIG. 3 is a schematic diagram indicting the alternative splicing of RGS9 pre-mRNA to form RGS9-1 and RGS9-2 mRNA (E1-E16=exons 1-16, E17A=exon 17A, E17B=exon 17b, E18=exon 18, and E19=exon 19);

FIG. 4 shows the levels of RGS9 splice variants in the blood of Parkinsonian mice described in Example 1. The mice were euthanized 3 days (D) after acute MPTP treatment, or 3 days (D) or 3 weeks (WK) after chronic MPTP administration;

FIG. 5 is a schematic diagram indicting the alternative splicing of AChE pre-mRNA to form AChE-R and AChE-S mRNA (E1=exon 1, E2=exon 2, E3=exon 3, E4=exon 4, E4′=exon 4′, E5=exon 5, and E6=exon 6);

FIG. 6 shows the levels of AChE splice variants in the blood of Parkinsonian mice described in Example 1. The mice were euthanized 3 days (D) after acute MPTP treatment, or 3 days (D) or 3 weeks (WK) after chronic MPTP administration;

FIG. 7 is a schematic diagram indicting the alternative splicing of Ania6 pre-mRNA to form Ania6 and Ania6a mRNA (E1-5=exons 1-5, E6=exon 6, E6′=exon 6′, E7=exon 7, E8=exon 8, and E9-12=exons 9-12);

FIG. 8 shows the levels of Ania6 splice variants in the blood of Parkinsonian mice described in Example 1. The mice were euthanized 3 days (D) after acute MPTP treatment, or 3 days (D) or 3 weeks (WK) after chronic MPTP administration;

FIG. 9 is a schematic diagram indicting the alternative splicing of NDUFS4 pre-mRNA to form NDUFS4 and NDUFS4-SV3 mRNA (E1=exon 1, E2=exon 2, E3=exon 3, E4=exon 4, and E5=exon 5); and

FIG. 10 shows the level of NDUFS4 splice variants in the brain and blood of Parkinsonian mice described in Example 1. The mice were euthanized 3 days (D) after acute MPTP treatment, or 3 days (D) or 3 weeks (WK) after chronic MPTP administration.

### DETAILED DESCRIPTION OF THE INVENTION

While this invention is susceptible to embodiments in many different forms, it is shown in the drawings and will herein be described in detail a preferred embodiment of the invention with the understanding that the present disclosure is to be considered as an exemplification of the principles of the invention and is not intended to limit the broad aspect of the invention to the embodiments illustrated.

The present invention provides a method for discovering biomarkers indicative of idiopathic neurodegeneration in a mammalian species that can be used to identify pre-symptomatic individuals within the species at risk for developing Parkinson's disease (PD) and other neurodegenerative diseases such as Alzheimer's disease. Early detection of these diseases could improve disease management and enable potential future neuroprotective therapies to be introduced at a stage when they would provide the greatest benefit. In addition, the biomarkers may be useful in identifying unknown environmental factors that play a role in the development of PD and other neurodegenerative diseases.

The method comprises (a) providing a test subject from the mammalian species having been exposed to an environmental factor that causes the neurodegenerative disease or having been positively diagnosed with the neurodegenerative disease; (b) obtaining RNA from a tissue of the test subject; (c) determining the amounts of a first splice variant mRNA of a precursor-messenger RNA (pre-mRNA) of a gene of the test subject, a second splice variant mRNA of the same pre-mRNA of the same gene and the total 18S RNA; (d) determining for the test subject a first ratio of the amount of first splice variant mRNA to the amount of the second variant mRNA or determining for the test subject a second ratio of the amount of the first splice variant mRNA to the amount of total 18S RNA; (e) obtaining a third ratio of a control subject of the amount of the first splice variant mRNA to the amount of the second splice variant mRNA or a fourth ratio of a control subject of the amount of the first splice variant mRNA to the amount of total 18S RNA; (f) comparing the first ratio to the third ratio to determine a first difference or comparing the second ratio to the fourth ratio to determine a second difference; and (g) identifying the first splice variant mRNA as a biomarker indicative of the neurodegenerative disease in the mammalian species if the first difference or the second difference is not zero, which can be positive or negative.

The present invention further discloses a biomarker indicative of an idiopathic neurodegenerative disease in a subject of a mammalian species, the subject having a first amount of a first splice variant mRNA of a precursor-messenger RNA (pre-mRNA) transcript of a gene in the mammalian subject, a second amount of a second splice variant mRNA of the same precursor-messenger RNA (pre-mRNA) transcript of the same gene and a total amount of 18S RNA, wherein the biomarker for the mammalian species comprises the first splice variant mRNA satisfying one of the conditions selected from the group consisting of: (a) a ratio of the amount of the first splice variant to the amount of the second splice variant of the mammalian subject having been exposed to an environmental factor that causes the neurodegenerative disease or having been positively diagnosed with the neurodegenerative disease is different from a ratio of the amount of the first splice variant to the amount of the second splice variant in a control subject who does not have the neurodegenerative disease; and (b) a ratio of the amount of the first splice variant to the amount of total 18S RNA of the mammalian subject having been exposed to an environmental factor known to cause the neurodegenerative disease or having been positively diagnosed with the neurodegenerative disease is different from a ratio of the amount of the first splice variant to the amount of total 18S RNA in a control subject who does not have the neurodegenerative disease. The difference can be positive of negative.

Examples of biomarkers indicative of neurodegenerative diseases in the present invention include but are not limited to splice variant mRNAs of pre-mRNAs from the genes FosB, RGS9, Ania6, AChE and NDUFS4. These biomarkers can be used in the diagnosis and prognosis of neurodegenerative diseases. They can also be used to screen for potential therapeutic agents for the treatment of neurodegenerative diseases.

There is no cure for these neurodegenerative diseases and, to date, there are no biomarkers to identify susceptible individuals. With new tools, such as those disclosed in the present invention, i.e. the discovery of splice variants of “at risk’ transcripts, we can identify presymptomatic individuals on the basis of their possessing an abnormal ratio of spliced isoforms from a single gene. Such detection is increasingly more important today as new neuroprotective agents are developed and can be used to combat disease in early stages. Furthermore, the elucidation of altered gene products sets the stage for the development of novel therapeutic strategies to target specific splicing isoforms.

Alternative Precursor-Messenger RNA (Pre-mRNA) Splicing

Pre-mRNA splicing is a regulated process to remove non-coding sequences called introns from the pre-mRNA. It occurs after gene transcription and prior to mRNA translation. Alternative pre-mRNA splicing is the process of differential inclusion or exclusion of regions of the pre-mRNA that allows a single transcript to produce one or more variants of RNA and protein products. While alternative splicing for many genes is found in normal individuals, alternative or regulated splicing may be the cause of pathogenic changes at RNA and protein levels. Alternative splice products have been identified in certain diseases, including experimental and clinical Parkinson's disease. In the present disclosure, we show that the changes in the levels of splice variants of certain genes, such as FosB, RGS9, Ania6, AChE and NDUFS4, are indicative of neurodegenerative disease.

Neurological Disease is Linked to Aberrant Splicing

Immediately after the discovery of RNA splicing, it was apparent that mistakes in and dysregulation of RNA processing could lead to disease. The magnitude of the contribution of such problems is only beginning to be understood. In order to understand how mistakes in splicing and alteration of its regulation leads to disease, it was necessary to first identify the essential components and characterize their function. It is clear that proper splicing in humans requires the recognition of weakly conserved splice sites at the 5′ and 3′ splice sites and the branch-point of the lariat intermediate. The identification of exons and introns involves five small ribonucleoprotein particles (snRNPs) and eighty or more proteins. Because the recognitions sequences for splicing are loosely defined, additional sequences referred to as enhancers and silencers are also required. There are several factors that regulate splicing by binding to these recognition sequences. The concentration, distribution, composition and state of modification of these regulatory factors determine whether they enhance or inhibit a particular splice site.

While some cases of neurodegenerative diseases are likely to be a result of genetic mutation, these cases are rare. The majority of cases of Alzheimer's disease (AD), the most common neurodegenerative disease, and Parkinson's disease (PD), the most common neurodegenerative movement disorder, are idiopathic. At least 90% of the cases of amyotrophic lateral sclerosis (ALS) are also sporadic. Since the vast majority of the cases of neurodegenerative disease are sporadic, it would be beneficial to determine the underlying mechanisms in order to identify biomarkers to characterize disease progression and to design treatments. What is meant by “idiopathic” or “sporadic” as used in the present disclosure and referring to neurodegenerative diseases is that the neurodegenerative disease arises spontaneously or from an obscure or unknown cause, which is different from the neurodegenerative diseases that are the results of genetic mutations. The terms “idiopathic” and “sporadic” are used interchangeably in the present disclosure.

The main problem with determining the basis of the sporadic forms of neurodegenerative disease is distinguishing which molecular changes are primary events and which are secondary. One thing which is known, however, is that oxidative stress and exposure to heavy metals, along with other cellular stresses, may cause a dysregulation (or loss of proper regulation) of splicing that alters the ratio of splice variants and produces disease. There are many known examples of neurodegeneration resulting from dysregulation of splicing. The loss of proper regulation is most likely due to changes in the regulatory factors brought about by environmental stimuli. It is likely that mistakes in splicing and a loss of its regulation is a common mechanism underlying neurodegenerative disease.

Many cancers and inherited diseases are associated with abnormalities in the regulation of splicing. There are numerous examples of changes in splicing associated with inherited neurological diseases (D'Souza et al., 1999, Missense and silent tau gene mutations cause frontotemporal dementia with parkinsonism-chromosome 17 type by affecting multiple alternative RNA splicing regulatory elements. Proc Natl Acad Sci U S A 96:5598-5603; Grabowski and Black, 2001, Alternative RNA splicing in the nervous system. Prog Neurobiol 65:289-308; Zhang et al., 2002, Region-specific alternative splicing in the nervous system: implications for regulation by the RNA-binding protein NAPOR. RNA 8:671-685). However, it is unclear how splicing regulation becomes disrupted in patients who do not inherit genetic defects. Some patients may acquire a genetic defect that affects splicing after exposure to environmental stimuli. It is also possible in other cases that exposure to stressful environmental stimuli activates new signaling pathways that result in a change in the concentration, localization, and/or modification of regulatory factors required for the maintenance of proper splicing. There are numerous examples of altered splice site selection in response to stresses such as pH change, osmotic or temperature shock, exposure to UV light or forced physical activity (see Stamm, 2002, Signals and their transduction pathways regulating alternative splicing: a new dimension of the human genome. Hum Mol Genet 11:2409-2416). One example is the translocation of SR regulatory factors from the nucleus to the cytoplasm in brain ischemia (Daoud et al., 2002, Ischemia induces a translocation of the splicing factor tra2-beta 1 and changes alternative splicing patterns in the brain. J Neurosci 22:5889-5899). The result of this change is a disruption of the normal regulated splicing of the interleukin-1β converting enzyme homologue 1 pre-mRNA. Similarly, an increase in the intracellular calcium concentration may result in a translocation of splicing factors. Based on the very limited number of studies addressing this problem, it is obvious that further research is needed to elucidate how splicing changes are initiated by environmental factors. The present invention discloses the gene expression changes that occur in mammalian subjects with PD, and possibly other neurodegenerative diseases, in which the ratio of one mRNA splice product to another mRNA splice product is altered. The amounts of mRNAs of these splice variants in a subject can be used as biomarkers of the disease process for neurodegenerative diseases such as PD. For idiopathic neurodegeneration, splice variants of RNA are better biomarkers than specific genes since it represents the molecular changes in gene expression responsible for the disease development when no genomic mutations are present.

Alternatively Spliced Transcripts Whose Expression Changes in Models of PD.

Some changes in gene expression that occur in animals and cell culture models of PD have been identified (Youdim et al., 2002, Early and late molecular events in neurodegeneration and neuroprotection in Parkinson's disease MPTP model as assessed by cDNA microarray; the role of iron. Neurotox Res 4:679-689; Mandel et al., 2003, Using cDNA microarray to assess Parkinson's disease models and the effects of neuroprotective drugs. Trends Pharmacol Sci 24:184-191). In these studies, cDNA microarrays are often used to identify an increase or decrease in RNA expression. However, the technique does not provide information about post-transcriptional modifications and thus the microarrays used in these studies only provide a partial view of the biological changes that occur in disease development.

We investigated whether transcripts that either increased or decreased in expression in these studies have alternatively spliced products. We used data from the putative alternative splice database and published results. Our search indicated that approximately 50% of the transcripts whose expression was increased or decreased in parkinsonism or in PD may have splice variants. Some of these transcripts are listed in Table 1.

Splice variants of α-synuclein, synphilin, syntaxin 8, parkin, FosB, RGS9-2 and Nurr1 are especially of interest since these genes have been associated with Parkinsonism in animals or PD in patients. In one study which investigated the expression of FosB and the Regulator of G protein signaling 9 (RGS9) in human PD patients, it was found that both RGS9-2 and ΔFosB protein levels were elevated in the striatum of these patients (Tekumalla P. K. et al., 2001, Elevated levels of DeltaFosB and RGS9 in striatum in Parkinson's disease. Biol Psychiatry 50:813-816). RGS9-2 and ΔFosB are the products of alternative splicing. The study, however, did not look at the FosB or RGS9 mRNAs.

TABLE 1
Gene Expression Data. Selected transcripts that have splice variants
appear in the table. A total of 98 oxidative stress (OS), 27 MPTP (mptp),
and 11 transcripts have been found to be altered in microarray studies
and studies on PD patients and animal models of Parkinsonism.

| Function | Name | Study | Change |
|---|---|---|---|
| Stress | Gtase | mptp | Down |
| | HemeOxygenase I | OS | Up |
| Receptors | IL-1 recept II | mptp | Up |
| | IL-2 rp-G | mptp | Up |
| | IL-10 | mptp | Up |
| | Ach N receptor | mptp | Up |
| ER/ubiquitin-like | U-like protein3 | OS | Up |
| | Torsin | OS | Up |
| Cell cycle | Cyclin B2 | mptp | Down |
| Apoptosis | BAX M, isoform-a | mptp | Up |
| | Apaf1 | OS | Up |
| PD-associated | Alpha-synuclein | mptp | Up |
| | RGS9-2 | PD | unknown |
| | Nurr 1 | PD | unknown |
| | Synphilin | PD | unknown |
| | Parkin | PD | unknown |
| | Syntaxin 8 | PD | unknown |

Splice Variant mRNA Transcripts as Biomarkers for Neurodegenerative Diseases

In an embodiment, the present invention provides a method for discovering biomarkers indicative of an idiopathic neurodegenerative disease in a mammalian species. The method comprises (a) providing a test subject from the mammalian species, the subject having been exposed to an environmental factor that causes the neurodegenerative disease or having been positively diagnosed with the neurodegenerative disease; (b) obtaining RNA from a tissue of the test subject; (c) determining the amounts of a first splice variant mRNA of a precursor-messenger RNA (pre-mRNA) of a gene of the test subject, a second splice variant mRNA of the same pre-mRNA of the same gene and the total 18S RNA; (d) determining for the test subject a first ratio of the amount of first splice variant mRNA to the amount of the second variant mRNA or determining for the test subject a second ratio of the amount of the first splice variant mRNA to the amount of total 18S RNA; (e) obtaining a third ratio of a control subject of the amount of the first splice variant mRNA to the amount of the second splice variant mRNA or a fourth ratio of a control subject of the amount of the first splice variant mRNA to the amount of total 18S RNA; (f) comparing the first ratio to the third ratio to determine a first difference or comparing the second ratio to the fourth ratio to determine a second difference; and (g) identifying the first splice variant mRNA as a biomarker indicative of the neurodegenerative disease in the mammalian species if the first difference or the second difference is not zero, which can be positive or negative. The mammalian species can be any species, such as but is not limited to mouse or human.

The exposure to the environmental factor can be unintentional or intentional. The exposure is unintentional if the subject is unintentionally exposed to the factor, such as the subject being exposed to the environmental factor in the environment. The subject may or may not be aware of the exposure. The exposure is intentional if the subject is exposed to the factor intentionally, such as intentionally treating the subject with the factor as demonstrated in the Parkinsonian mouse model described in Example 1 below. In general, the environmental factor is a toxic chemical which is known to cause a neurodegenerative disease. An example of such an environmental factor is 1-methyl-4-phenyl-1,2,3,6-tetrahydropyridin (MPTP), which is used in the present disclosure to induce PD in experimental mice. Many herbicides are pesticides are also known to cause neurodegenerative diseases. In addition to chemicals, the environmental factor can also be a mechanical force to the central nervous system of the subject.

The exposure to the environmental factor can be acute or chronic. The exposure time for an acute or chronic exposure depends on the life spans of the species of the mammalian subject. Acute exposure generally means a single or multiple exposures to the factor within a short period of time, such as minutes or a small number of (lays (e.g., 3 days) in mice, or minutes, days and weeks (e.g., 4 weeks) in human. Chronic exposure generally means continual or intermittent exposure for a prolong period of time such as weeks (e.g., 3 weeks) in mice, or months (e.g., 6 months) and years (e.g., 1 year or more) in human. The acute exposure reflects immediate changes in gene expression that occur shortly after the exposure. The chronic exposure represents more long-term exposure to the factor. Thus, some of these biomarkers are useful in identifying immediate changes after exposure to the environmental factor while other biomarkers are indicative of changes that occur with time.

The tissue in which the RNA is obtained can be any tissue from the subject, such as but are not limited to brain, blood, and cerebral spinal fluid (CSF). In a preferred embodiment, the tissue is blood or CSF.

The neurodegenerative disease can be any neurodegenerative disease. For illustrative purposes, the present invention is demonstrated using a well established 1-methyl-4-phenyl-1,2,3,6-tetrahydropyridine (MPTP) mouse model in which experimental mice were treated with MPTP to induce PD in the mice (Petroske et al., 2001, Mouse model of parkinsonism: A comparison between subacute MPTP and chronic MPTP/probenecid treatment, Neuroscience 106(3): 589-601). However, the present invention should also be able to extend to other neurodegenerative diseases, such as Alzheimer's diseases, by using the appropriate environmental factor which is known to cause that neurodegenerative disease.

In another embodiment, the present invention discloses a biomarker indicative of an idiopathic neurodegenerative disease in a subject of a mammalian species, the subject having a first amount of a first splice variant mRNA of a precursor-messenger RNA (pre-mRNA) transcript of a gene in the mammalian subject, a second amount of a second splice variant mRNA of the same precursor-messenger RNA (pre-mRNA) transcript of the same gene and a total amount of 18S RNA, wherein the biomarker for the mammalian species comprises the first splice variant mRNA satisfying one of the conditions selected from the group consisting of: (a) a ratio of the amount of the first splice variant to the amount of the second splice variant of the mammalian subject having been exposed to an environmental factor that causes the neurodegenerative disease or having been positively diagnosed with the neurodegenerative disease is different from a ratio of the amount of the first splice variant to the amount of the second splice variant in a control subject who does not have the neurodegenerative disease; and (b) a ratio of the amount of the first splice variant to the amount of 18S RNA of the mammalian subject having been exposed to an environmental factor that causes the neurodegenerative disease or having been positively diagnosed with the neurodegenerative disease is different from a ratio of the amount of the first splice variant to the amount of total 18S RNA in a control subject who does not have the neurodegenerative disease. The difference can be positive or negative.

RNA can be obtained from the tissue, such as but is not limited to blood or cerebrospinal fluid (CSF), of a mammalian subject, particularly human, using any suitable method. Preferably, venous blood or CSF is drawn in accordance with methods known in the art. Preferably, the blood or cerebrospinal fluid is kept cold, e.g., on ice, until use. When using plasma, blood should not be permitted to coagulate prior to separation of the cellular and acellular blood components. Preferably, within 30 min of drawing blood, serum is separated by centrifugation, e.g., at 1100×g at 4° C. Serum or plasma can be frozen, for example, at −70° C., after separation from the cellular component of blood. When using frozen blood plasma or serum, the frozen plasma or serum can be slowly thawed or rapidly thawed, for example in a water bath at 37° C., and RNA is extracted without delay.

RNA can be extracted from the blood or CSF using any suitable method, such as the methods set forth below in Example 2. Desirably, the RNA is extracted as soon as possible so as to minimize degradation of the RNA. Examples of suitable extraction methods include, but are not limited to, gelatin, silica, glass bead, diatom, guanidinum thiocyanate acid-phenol, guanidinium thiocyanate acid, centrifugation through cesium chloride or a similar gradient, phenol-chloroform, or a commercially available kit, such as the Promega SV total RNA isolation system (Promega, Madison, Wis.), the Perfect RNA Total RNA Isolation Kit (Five Prime-Three Prime, Inc., Boulder, Colo.), or the TRI Reagent BD kit (Molecular Research Center, Inc., Cincinnati, Ohio). Alternatively, extraction can be performed using probes that specifically hybridize to a particular RNA, in particular isolation methods dependent thereupon, e.g., chromatographic methods and methods for capturing RNA hybridized to the probes.

The extracted RNA is then amplified, either after conversion into cDNA or directly, using in vitro amplification methods. Examples of amplification methods include, but are not limited to, reverse transcriptase-polymerase chain reaction (RT-PCR; see, e.g., U.S. Pat. Nos. 4,683,195; 4,683,202; and 4,800,159 for PCR techniques, generally), ligase chain reaction, DNA signal amplification, amplifiable RNA reporters, Q-beta replication, transcription-based amplification, boomerang DNA amplification, strand displacement activation, cycling probe technology, isothermal nucleic acid sequence-based amplification, and other self-sustained sequence replication assays.

Preferably, the RNA is amplified using RT-PCR. RNA can be reverse-transcribed into cDNA using Moloney murine leukemia virus (MMLV) reverse transcriptase (Promega), a reaction buffer supplied by the manufacturer, dNTPs, random hexameric oligonucleotide primers, and RNAsin (Promega). Reverse transcription is typically performed by incubation at room temperature for 10 min, followed by incubation at 37° C. for 1 hr. Alternatively, reverse transcription can be performed in accordance with the method of Rajagopal et al., 1995 (Epidermal growth factor expression in human colon and colon carcinomas: anti-sense epidermal growth factor receptor RNA down-regulates the proliferation of human colon cancer cells Int. J. Cancer 62: 661-667) or Dahiya et al., 1996 (Differential gene expression of transforming growth factors alpha and beta, epidermal growth factor, keratinocyte growth factor, and their receptors in fetal and adult human prostatic tissues and cancer cell lines, Urology 48: 963-970). Amplification oligonucleotide primers are selected to be specific for amplifying the nucleic acid of interest. Therefore, the oligonucleotide primers need to be of sufficient length to achieve specificity, yet not so long as to affect adversely the efficiency of the reaction. Optimally, the primers are from about 18 to about 21 nucleotides in length.

The amplified product then can be separated, such as by gel electrophoresis (e.g., 1-2% agarose gel), and visualized (e.g., ethidium bromide staining) in accordance with methods known in the art. Alternatively, capillary electrophoresis, amplification using biotinylated or otherwise modified primers, nucleic acid hybridization using specific, detectably-labeled probes, such as fluorescently, radioactively, or chromogenically labeled probes, Southern blot, Northern blot, electrochemiluminescence, laser-induced fluorescence, reverse dot blot, or high performance liquid chromatography can be used. Detection can be qualitative or quantitative.

Using the method described above, splice variant mRNAs of various pre-mRNAs have been examined in mouse blood samples from MPTP-treated mice. Splice variant mRNAs from 5 separate genes have been identified as biomarkers indicative of idiopathic neurodegenerative diseases. These genes include FosB, RGS9, Ania6, AChE and NDUFS4. Some of the ratios of the splice variants of these pre-mRNA transcripts to 18S rRNA standard change in the blood of the MPTP-treated mice compared to vehicle-treated mice. These splice variants can be used as biomarkers for idiopathic neurodegenerative diseases such as PD. In addition, the Ania6, AChE and NDUFS4 splice variant ratios change in the blood of MPTP-treated mice compared to vehicle-treated mice. Therefore, the ratios of these splice variants can be used as biomarkers for neurodegenerative diseases such as PD.

The following is a summary of using these splice variants as biomarkers for acute or chronic Parkinson's disease:
1. ΔFosB mRNA: The ratio of ΔFosB mRNA/18S RNA decreases after chronic MPTP treatment (data shown in FIG. 2) and therefore can be used as a persistent marker for chronic exposure to environmental factors that lead to neurodegeneration. Thus, the present invention discloses a biomarker indicative of chronic idiopathic Parkinson's disease in a test mammalian subject comprising ΔFosB mRNA wherein a decrease in the ratio of the amount of the ΔFosB mRNA to the amount of total 18S RNA in the test subject as compared to a control without the disease is indicative of the disease.
2. FosB mRNA: The ratio of FosB mRNA/18S RNA decreases after acute MPTP treatment (data shown in FIG. 2) and therefore can be used as an early marker for exposure to environmental factors that lead to neurodegeneration. Thus, the present invention discloses a biomarker indicative of acute idiopathic Parkinson's disease in a test mammalian subject comprising FosB mRNA wherein a decrease in the ratio of the amount of the FosB mRNA to the amount of total 18S RNA in the test subject as compared to a control without the disease is indicative of the disease.
3. RGS9-2 mRNA: The ratio of RGS9-2 mRNA/18S RNA decreases after acute MPTP treatment (data shown in FIG. 4) and therefore can be used as an early marker for exposure to environmental factors that lead to neurodegeneration. The RGS9-2 mRNA/RGS9-1 mRNA decreases after chronic MPTP treatment and remains lower for 3 weeks and therefore can be used as a persistent marker for chronic exposure to environmental factors that lead to neurodegeneration (data shown in FIG. 4). Thus, the present invention discloses a biomarker indicative of acute idiopathic Parkinson's disease in a test mammalian subject comprising RGS9-2 mRNA wherein a decrease in the ratio of the amount of the RGS9-1 mRNA or a decrease in the ratio of the RGS9-2 mRNA to the amount of total 18S RNA in the test subject as compared to a control without the disease is indicative of the disease.
4. AChE-R mRNA: The ratio of AChE-R mRNA/AChE-S mRNA and AChE-R mRNA/18S RNA increases after acute MPTP treatment (data shown in FIG. 6) and therefore can be used as an early marker for exposure to environmental factors that lead to neurodegeneration. Thus, the present invention discloses a biomarker indicative of acute idiopathic Parkinson's disease in a test mammalian subject comprising AChE-R mRNA wherein an increase in the ratio of the amount of AChE-R mRNA to the amount AChE-S mRNA or an increase in the ratio of the amount of AChE-R mRNA to the amount of total 18S RNA in the test subject as compared to a control without the disease is indicative of the disease.

The ratio of AChE-R mRNA/AChE-S mRNA decreases and AChE-R mRNA/18S RNA decreases after chronic MPTP treatment and remains lower for 3 weeks (data shown in FIG. 6) and therefore can be used as a persistent marker for chronic exposure to environmental factors that lead to neurodegeneration. Thus, the present invention discloses a biomarker indicative of chronic idiopathic Parkinson's disease in a test mammalian subject comprising AChE-R mRNA wherein a decrease in the ratio of the amount of ACHE-R mRNA to the amount AChE-S mRNA or a decrease in the ratio of the amount of ACHE-R mRNA to the amount of total 18S RNA in the test subject as compared to a control without the disease is indicative of the disease.
5. AChE-S mRNA: The ratio of AChE-S mRNA/18S RNA increases after chronic MPTP treatment (data shown in FIG. 6) and therefore can be used as a marker for chronic exposure to environmental factors that lead to neurodegeneration. Thus, the present invention discloses a biomarker indicative of chronic idiopathic Parkinson's disease in a test mammalian subject comprising AChE-S mRNA wherein an increase in the ratio of the amount of AChE-S mRNA to the amount of total 18S RNA in the test subject as compared to a control without the disease is indicative of the disease.
6. Ania6a mRNA: The ratio of Ania6a mRNA/Ania6 mRNA ratio increases and the Ania6 mRNA/18S RNA decreases after chronic MPTP treatment and remains lower for 3 weeks (data shown in FIG. 8) and therefore can be used as a persistent marker for chronic exposure to environmental factors that lead to neurodegeneration. Thus, the present invention discloses a biomarker indicative of chronic idiopathic Parkinson's disease in a test mammalian subject comprising Ania6a mRNA wherein an increase in the ratio of the amount of Ania6a mRNA to the amount Ania6 mRNA or an increase in the ratio of the amount of Ania6a mRNA to the amount of total 18S RNA in the test subject as compared to a control without the disease is indicative of the disease.

The ratio of Ania6a mRNA/18S RNA increases after chronic MPTP treatment (data shown in FIG. 8) and therefore can be used as a marker for chronic exposure to environmental factors that lead to neurodegeneration. Thus, the present invention discloses a biomarker indicative of chronic idiopathic Parkinson's disease in a test mammalian subject comprising Ania6 mRNA wherein an increase in the ratio of the amount of Ania6 mRNA to the amount of total 18S RNA in the test subject as compared to a control without the disease is indicative of the disease.
7. NDUFS4-SV3 mRNA: The ratio of NDUFS4-SV3 mRNA/NDUFS4 mRNA and NDUFS4-SV3 mRNA/18S RNA decreases after chronic MPTP treatment and remains lower for 3 weeks (data shown in FIG. 10) and therefore can be used as a persistent marker for chronic exposure to environmental factors that lead to neurodegeneration. Thus, the present invention discloses a biomarker indicative of chronic idiopathic Parkinson's disease in a test mammalian subject comprising NDUFS4-SV3 mRNA wherein a decrease in the ratio of the amount of NDUFS4-SV3 mRNA to the amount NDUFs4 mRNA or a decrease in the ratio of the amount of NDUFS4-SV3 mRNA to the amount of total 18S RNA in the test subject as compared to a control without the disease is indicative of the disease.
8: NDUFS4 mRNA: The ratio of NDUFS4 mRNA/18S RNA decreases after chronic MPTP treatment and therefore can be used as a marker for chronic exposure to environmental factors that lead to neurodegeneration. Thus, the present invention discloses a biomarker indicative of chronic idiopathic Parkinson's disease in a test mammalian subject comprising NDUFS4 mRNA wherein a decrease in the ratio of the amount of NDUFS4 mRNA to the amount of total 18S RNA in the test subject as compared to a control without the disease is indicative of the disease.

While the above examples of biomarkers were derived from mice, the methods of the present disclosure can be applied to any mammalian species, including human. The biomarkers in one mammalian species may or may not be the same as those in another species. In other words, a specific splice variant mRNA identified as a biomarker for one species may or may not be a biomarker for another species. In addition, even if two species have the same splice variant mRNA as a biomarker, the direction of change of the ratio of the splice variant mRNA biomarker relative to another splice variant mRNA of the same pre-mRNA or relative to the total 18S RNA in one mammalian species may or may not be the same as that in another mammalian species. Similarly, the biomarkers in a tissue from a mammalian species may or may not be the same as those in another tissue within the same mammalian species. The splice variant mRNA biomarker in one tissue from a species can be the same or different in another tissue from the same species, or the direction of change of the splice variant mRNA biomarker relative to another splice variant mRNA of the same pre-mRNA or to the total 18S RNA in one tissue of a species can be different from that of another tissue within the same species.

### EXAMPLES

The following examples serve to illustrate the present invention and are not intended to limit its scope in any way.

### Example 1

1-methyl-4-phenyl-1,2,3,6-tetrahydropyridin (MPTP) Mouse Model for PD

Mice were treated with MPTP, either acutely, which depletes the substantia nigra of approximately 50% of the dopamine cells, or chronically with a low dose MPTP treatment, which can kill as many as 80-90% of the dopaminergic neurons (E. Petroske, G. E. Meredith, S. Callen, S. Totterdell, Y. S. Lau, 2001, Mouse model of Parkinsonism: a comparison between subacute MPTP and chronic MPTP/probenecid treatment. Neuroscience 106, 589). Male C57B1/6 mice that weigh 18-22 g were injected with MPTP for the experiments. For the acute procedure, mice were injected with 20 mg/kg MPTP (in saline), 4 times, every 2 hours, and then euthanized 3 days later. For the chronic, low-dose procedure, mice were injected twice each week, at 3.5-day intervals, for 5 weeks with MPTP (25 mg/kg, s.c) and probenecid (250 mg/kg, i.p.). Mice were then euthanized either at 3 days or 3 weeks after treatment. Two time periods were selected in order to characterizing splicing events in the blood over time. Controls for the acute group include mice treated with saline and, for the chronic experiments, mice treated with probenecid or saline.

RNA can be obtained from brain tissue or cerebral spinal fluid (CSF) as described in U.S. patent application Ser. No.: 11/215,842.

### Example 2

Obtaining Blood from Mammalian Subjects

Blood specimens for assaying RNA from the mice described in Example 1 above were obtained as follows. Mice were sacrificed by cervical dislocation and the heart lacerated to release the blood. Approximately 50 μl of 0.05 M EDTA, pH 8.0 was added to the chest cavity to prevent the blood from coagulating. Blood was collected in a sterile 1 ml syringe that was initially rinsed in 0.50 M EDTA pH 8.0.

Blood specimens may be drawn from human or larger mammalian subjects in PAXgene Blood RNA tubes (Qiagen Inc., Valentia, Calif.) since these tubes contain RNA protection agents. Whole blood may be obtained and frozen at −80° C. until assayed. Frozen blood (200λ) are thawed on ice. TRI Reagent BD Supplement (Sigma Aldrich, St. Louis, Mo.), 0.75 ml, is added to the blood. Then 20λ of 5N Acetic Acid is added and the sample are incubated at room temperature (RT) with gentle mixing for 5 minutes. Chloroformn (0.2 ml) is then added to samples, the samples are hand-shaken vigorously for 15 seconds, and incubated at RT for 5 minutes. Samples are centrifuged at 12,000×g for 15 minutes at 4° C. The aqueous supernatant is transferred to a fresh ependorf test tube and 1λ glycogen is added to the sample. Isopropanol, 0.5 ml, is added to the sample. The sample is then gently shaken and incubated 10 minutes at RT. Samples are centrifuged at 12,000×g for 8 minutes at 4° C. The supernatant is removed and 1 ml of ethanol is added to the pellet. The sample is centrifuged at 7,500×g for 5 minutes at 4° C. The supernatant is removed. The pellet is air dried. The pellet is resuspended in 30λ of sterile warm distilled H₂O and incubated 10 minutes at 65° C. The samples thus obtained which contain RNA are then stored in aliquots at −80° C.

### Example 3

Assay for Total 18S RNA

RNA from blood samples obtained as described in Example 2 was separated on a 1.8% agarose gel and stained with 5 μl of ethidium bromide (10 mg/ml stock) diluted into 10 ml of a TAE buffer (pH 8.5 40 mM Tris.acetate, 2 mM Na₂EDTA.2H₂O. After staining, the RNA was visualized on a UV light box and the 18S RNA band was quantified. The image was captured using a Kodak EDAS290 system (Kodak, Rochester, N.Y.) and the Kodak 1D Image Analysis software (Kodak, Rochester, N.Y.) was used to quantify. Other similar cameras and analysis softwares will also work.

### Example 4

PCR Conditions for Amplification of FosB RGS9 AchE, Ania6 and NDUFS4 Splice Variants

Splice variant mRNA was assayed using reverse transcriptase polymerase chain reactions (RT-PCR) as described in U.S. patent application Ser. No.: 11/215,842. PCR conditions for amplification of FosB, RGS9, AchE, Ania6 and NDUFS4 are the same as disclosed in U.S. patent application Ser. No.: 11/215,842 except different kits were used for FosB and RGS9 compared to the other 3 transcripts.

FosB (SYBR green, Applied Biosystems, Foster City, Calif.)

1. 95° C. 8 minutes
2. 95° C. 30 seconds
3. 57° C. 30 seconds
4. 72° C. 30 seconds
5. Repeat from step 2 35 times
6. 72° C. 10 minutes
   RGS9 (SYBR green, Applied Biosystems, Foster City, Calif.)
1. 95° C. 8 minutes
2. 95° C. 30 seconds
3. 54° C. 30 seconds
4. 72° C. 30 seconds
5. Repeat from step 2 35 times
6. 72° C. 10 minutes
   AChE (go Taq Green, Promega, Madison, Wis.)
1. 95° C. 5 minutes
2. 95° C. 30 seconds
3. 61° C. 30 seconds
4. 72° C. 30 seconds
5. Repeat from step 2 30 times
6. 72° C. 5 minutes
   NDUFS4 (go Taq Green, Promega, Madison, Wis.)
1. 95° C. 5 minutes
2. 95° C. 30 seconds
3. 64° C. 30 seconds
4. 72° C. 30 seconds
5. Repeat from step 2 30 times
6. 72° C. 5 minutes
   Ania (go Taq Green, Promega, Madison, Wis.
1. 95° C. 5 minutes
2. 95° C. 30 seconds
3. 57° C. 30 seconds
4. 72° C. 30 seconds
5. Repeat from step 2 35 times
6. 72° C. 5 minutes
   Primers used in the PCR Assays:

FosB: Forward primer Exon 4, Reverse primer Exon 5

| | |
|---|---|
| Forward | |
| 5′ AAA AGG CAG AGC TGG AGT CGC 3′ | (SEQ ID NO:1) |
| Reverse | |
| 5′ GTA CGA AGG GCT AAC AAC GG 3′ | (SEQ ID NO:2) |
| RGS9 1: | |
| Forward- Exon 16, Reverse- Exon 17 | |
| RGS9 2: | |
| Forward- Exon 17, Reverse- Exon 18 | |
| RGS9 1 | |
| Forward | |
| 5′ GAT TCT TAC GCA CGC TAT TTG A 3′ | (SEQ ID NO:3) |
| Reverse | |
| 5′ AGC TTG CTC ATG ACT GGG TG 3′ | (SEQ ID NO:4) |
| RGS9 2 | |
| Forward | |
| 5′ GGC AGC TGG AAG AAG AAG AGA A 3′ | (SEQ ID NO:5) |
| Reverse | |
| 5′ GAG GGC TCT CTG TTC TCA GTG A 3′ | (SEQ ID NO:6) |

AChE-R: Primers in Exon 3 (Forward and Exon 5 (Reverse)

| | |
|---|---|
| AChE-R | |
| Forward | |
| 5′ CCC CAA TGA CCC TCG AGA CT 3′ | (SEQ ID NO:7) |
| Reverse | |
| 5′ CCT CCT TCC AAC CCT TGC C 3′ | (SEQ ID NO:8) |

AChE-S: Primers in Exon 3 (Forward) and Exon 6 (Reverse)

| | |
|---|---|
| AChE-S | |
| Forward | |
| 5′ TCT TTG AAC ACC GTG CCT CC 3′ | (SEQ ID NO;9) |
| Reverse | |
| 5′ CTC CGC CTC GTC CAG AGT AT 3′ | (SEQ ID NO:10) |

Ania6:
Forward primer Exon 5, Reverse primer Exon 7

| | |
|---|---|
| Ania6 | |
| Forward | |
| 5′ TCA AGG CAG AGA GGA GGG TG 3′ | (SEQ ID NO:11) |
| Reverse | |
| 5′ GAA AGC GAA CAA AGA CAT TGG TT 3′ | (SEQ ID NO:12) |

Ania6a: Foward primer Intron 6, Reverse primer Exon 7

ANIA6a

| | |
|---|---|
| Forward | |
| 5′ TGC TGT GGG GAA GTG GTT AG 3′ | (SEQ ID NO:13) |
| Reverse | |
| 5′ GAA AGC GAA CAA AGA CAT TGG TT 3 | (SEQ ID NO:14) |

NDUFS4: Forward primer in Exon 1, Reverse primer spans Exon 3 and 4

| | |
|---|---|
| NDUFS4 | |
| Forward | |
| 5′ TGG GGC GAA GGG CAA TGG 3′ | (SEQ ID NO:15) |
| Reverse | |
| 5′ TGG AGA GGG GGT CAG CGG T 3′ | (SEQ ID NO:16) |

### Example 5

Splice Variant mRNAs of FosB as Biomarkers Indicative of Neurodegenerative Disease

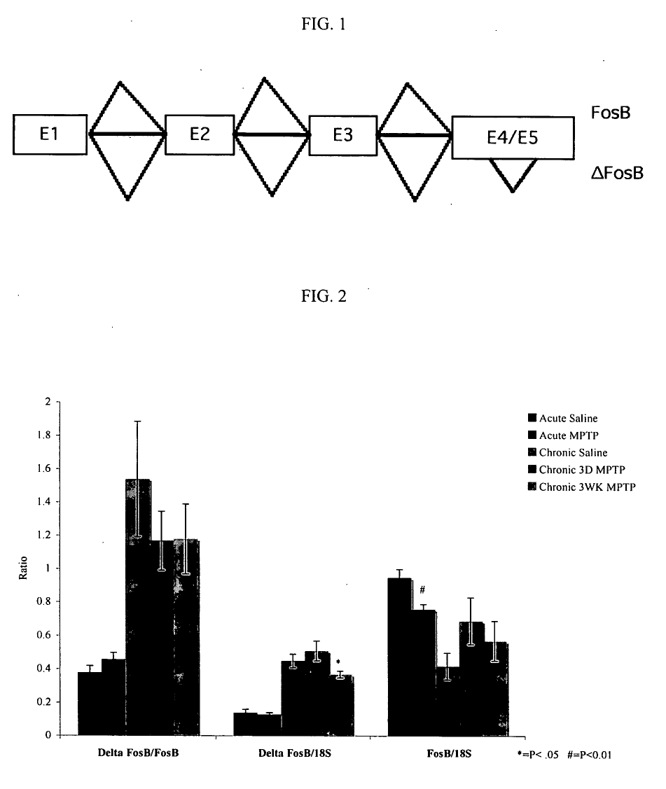

The gene for the transcription factor FosB produces two mRNAs by intron retention or splicing (FIG. 1). The intron-retained transcript produces full length FosB protein and the intron-spliced transcript produces ΔFosB. ΔFosB is a truncated protein that is missing its carboxyl-terminus and is more stable than FosB (Tekumalla et al., 2001, Elevated levels of DeltaFosB and RGS9 in striatum in Parkinson's disease. Biol Psychiatry 50, 813). Our data (see FIG. 2) demonstrate that the FosB mRNA/18S RNA decreases after acute MPTP treatment and therefore can be used as an early marker for exposure to environmental factors that lead to neurodegeneration. In addition, the ΔFosB mRNA/18S RNA decreases after chronic MPTP treatment and therefore can be used as a persistent marker for chronic exposure to environmental factors that lead to neurodegeneration.

### Example 6

Splice Variant mRNAs of RGS9 as Biomarkers Indicative of Neurodegenerative Disease

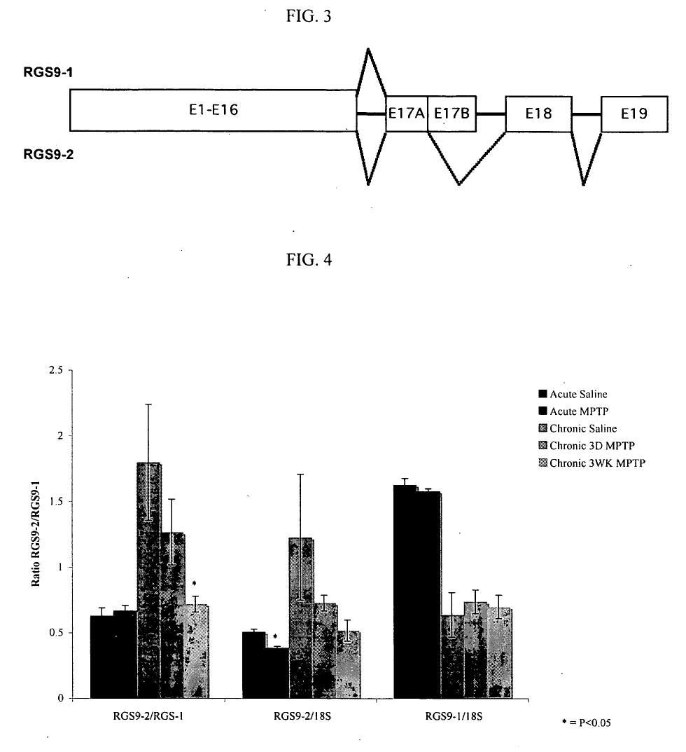

RGS9 is a regulator of G-protein signaling. The RGS9-2 protein is enriched in the striatum, whereas RGS9-1 protein is enriched in the retina (Zhang et al., 1999, Structure, alternative splicing, and expression of the human RGS9 gene. Gene 240, 23). Both transcripts of RGS9 contain the constitutively spliced exons 1-16 (FIG. 3). RGS9-1 mRNA also contains the entire exon 17 (A and B), whereas RGS9-2 contains exon 17A and exons 18 and 19 due to the use of an internal splice donor site (FIG. 4). Our data (FIG. 4) demonstrates that the RGS9-2 mRNA/18S RNA decreases after acute MPTP treatment and therefore may be used as an early marker for exposure to environmental factors that lead to neurodegeneration. In addition, the RGS9-2 mRNA/RGS9-1 decreases after chronic MPTP treatment and remains lower for 3 weeks and therefore can be used as a persistent marker for chronic exposure to environmental factors that lead to neurodegeneration.

### Example 7

Splice Variant mRNAs of ACHE as Biomarkers Indicative of Neurodegenerative Disease

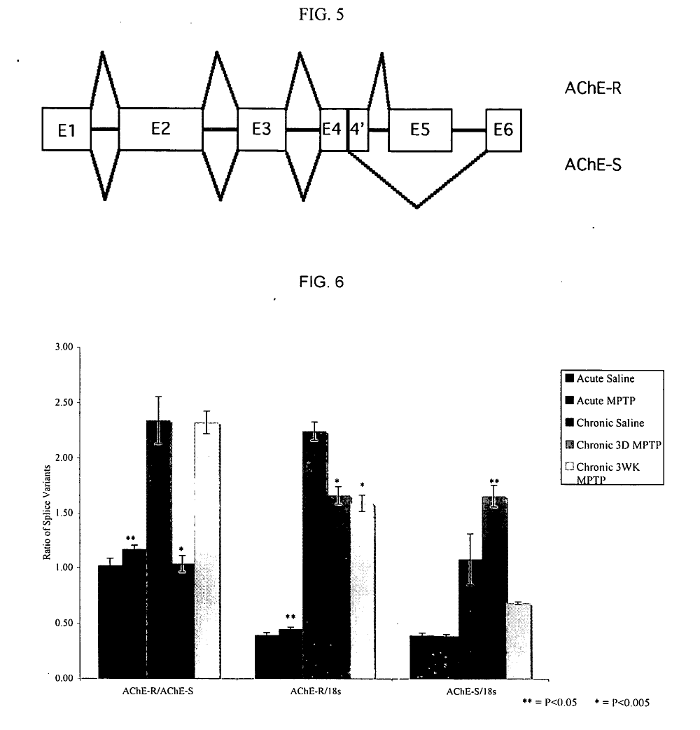

We investigated whether the amount of the splice variants of acetylcholinesterase (AChE) changed after MPTP toxin treatment. AChE hydrolyzes acetylcholine (ACh) and thereby functions to terminate Ach-mediated neurotransmission. In many neuropathologies, the characteristics of AChE are modified in the central nervous system. We looked at the AChE-S and ACHE-R splice variants. The AChE-S mRNA produces the canonical synaptic tetrameric form of the enzyme. AChE-S is formed by skipping exon 5 such that the final mRNA is composed of exons 1-4 and exon 6 (FIG. 5). The protein produced from this transcript has a cysteine-containing carboxyl-terminus that allows for the formation of the tetramer. AChE-R, produces a read-through transcript in which a downstream splice donor is spliced to the intron 4 3′ splice site such that exon 5 is included in the mRNA (FIG. 6). This produces a protein with a different carboxyl-terminus than the synaptic form. Importantly, the cysteine-containing region of the protein that is needed for tetramer formation is absent. This results in a protein that remains a monomer and thus soluble. ACHE-R is associated with neurodegeneration and stress-associated disorders (Meshorer and Soreq, 2006, Virtues and woes of AChE alternative splicing in stress-related neuropathologies. Trends Neurosci 29, 216). Our data (see FIG. 6) demonstrate that the AChE-R mRNA/AChE-S mRNA and AChE-R mRNA/18S RNA increases after acute MPTP treatment and therefore can be used as an early marker for exposure to environmental factors that lead to neurodegeneration. In addition, the AChE-R mRNA/AChE-S mRNA decreases and the AChE-S mRNA/18S RNA increases after chronic MPTP treatment and therefore may be used as a marker for chronic exposure to environmental factors that lead to neurodegeneration. The AChE-R mRNA/18S RNA decreases and remains lower for 3 weeks after chronic MPTP treatment and therefore can be used as a persistent marker for chronic exposure to environmental factors that lead to neurodegeneration.

### Example 8

Splice Variant mRNAs of Ania6 as Biomarkers Indicative of Neurodegenerative Disease

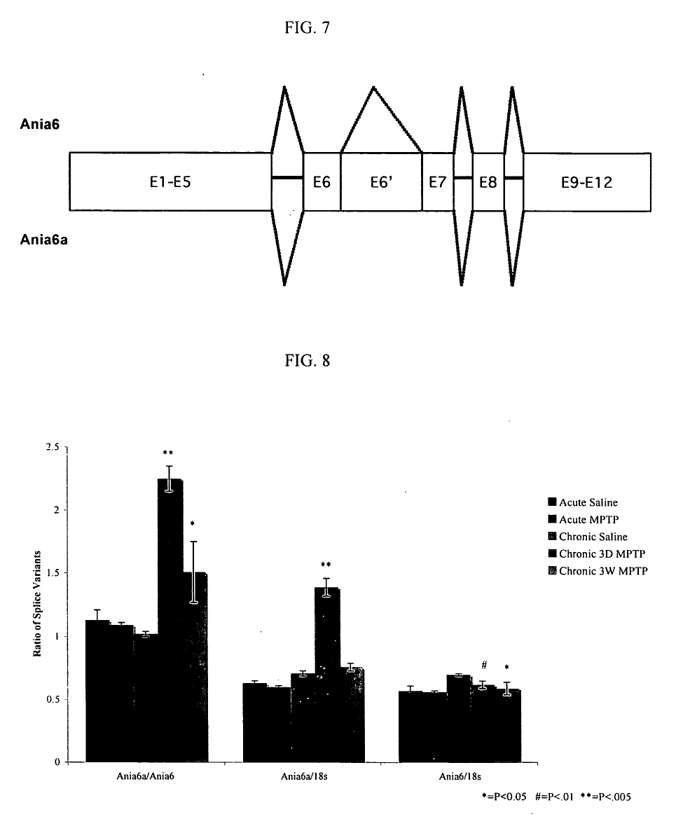

We also examined the two splice variants produced from the Ania 6 pre-mRNA. The Ania 6 gene encodes a cyclin, and its expression is induced in the striatum by dopamine stimulation (Berke et al., 2001, Dopamine and glutamate induce distinct striatal splice forms of Ania-6, an RNA polymerase II-associated cyclin. Neuron 32, 277). The longer splice variant, Ania6, is produced by retention of intron 6′, whereas the shorter splice variant, Ania6a, is produced when intron 6′ is spliced (FIG. 7). The Ania 6 protein contains a carboxyl-terminus with an arginine/serine-rich domain that is absent from the Ania 6a protein. This region of the protein is important for the association between the Ania 6 protein and splicing factors in the nucleus. To date, no study has examined the Ania6 splice variants in a model for a neurodegenerative disease. As shown in FIG. 8, the Ania6a mRNA/Ania6 mRNA ratio increases and the Ania6 mRNA/18S RNA decreases after chronic MPTP treatment and remains lower for 3 weeks and therefore can be used as a persistent marker for chronic exposure to environmental factors that lead to neurodegeneration. The Ania6a mRNA/18S RNA increases after chronic MPTP treatment and therefore can be used as a marker for chronic exposure to environmental factors that lead to neurodegeneration.

### Example 9

Splice Variant mRNAs of NDLFS4 as Biomarkers Indicative of Neurodegenerative Disease

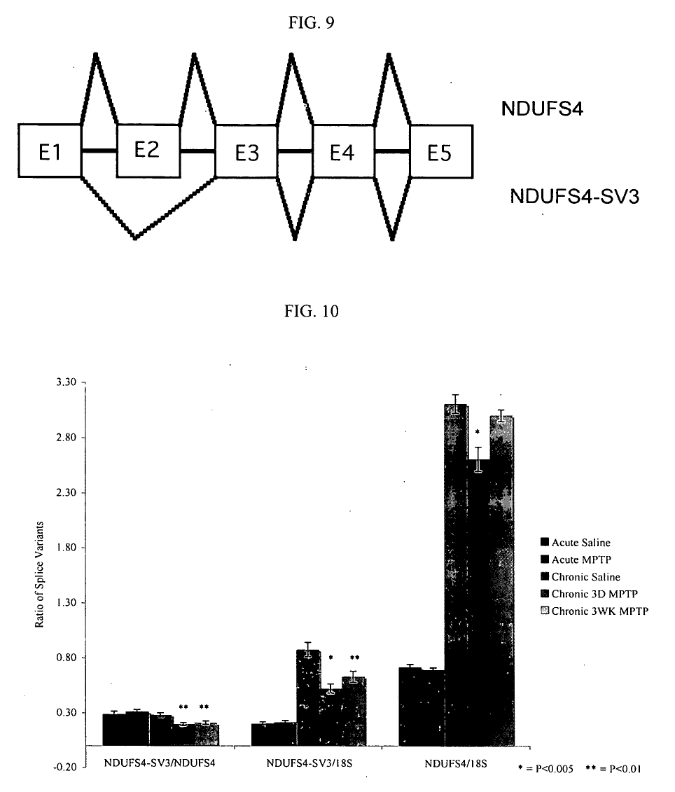

We also investigated the splicing of NDUFS4 (NADH-ubiquinone oxidoreductase Fe-S protein 4), a gene encoding a subunit of NADH of mitochondrial complex I because of the putative role complex I plays in the development of Parkinson's disease (Papa, 2002, The NDUFS4 nuclear gene of complex I of mitochondria and the cAMP cascade. Biochim. Biophys. Acta 1555, 147; Hirst et al, 2003, The nuclear encoded subunits of complex I from bovine heart mitochondria. Biochim. Biophys. Acta 1604, 135). The longer splice variant, NDUFS4, is produced by the splicing of 4 introns, whereas the shorter splice variant, NDUFS4-SV3, is produced when exon 2 is skipped (Petruzzella, et al., 2005, Mutations in the NDUFS4 gene of mitochondrial complex I alter stability of the splice variants. FEBS Letters 579, 3770; FIG. 9). To date, no study has examined the NDUFS4 splice variants in a model for a neurodegenerative disease, making our observations both novel and exciting. As shown in FIG. 10, the NDUFS4-SV3 mRNA/NDUFS4 mRNA and NDUFS4-SV3 mRNA/18S RNA decreases after chronic MPTP treatment and remains lower for 3 weeks and therefore can be used as a persistent marker for chronic exposure to environmental factors that lead to neurodegeneration. The NDUFS4 mRNA/18S RNA decreases after chronic MPTP treatment and therefore can be used as a marker for chronic exposure to environmental factors that lead to neurodegeneration

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

It is understood that, given the above description of the embodiments of the invention, various modifications may be made by one skilled in the art. Such modifications are intended to be encompassed by the claims below.

### REFERENCES

D'Souza et al., 1999, Missense and silent tau gene mutations cause frontotemporal dementia with parkinsonism-chromosome 17 type by affecting multiple alternative RNA splicing regulatory elements. Proc Natl Acad Sci U S A 96:5598-5603

Grabowski and Black, 2001, Alternative RNA splicing in the nervous system. Prog Neurobiol 65:289-308

Zhang et al., 2002, Region-specific alternative splicing in the nervous system: implications for regulation by the RNA-binding protein NAPOR. RNA 8:671-685

Stamm, 2002, Signals and their transduction pathways regulating alternative splicing: a new dimension of the human genome. Hum Mol Genet 11:2409-2416

Youdim et al., 2002, Early and late molecular events in neurodegeneration and neuroprotection in Parkinson's disease MPTP model as assessed by cDNA microarray; the role of iron. Neurotox Res 4:679-689

Mandel et al., 2003, Using cDNA microarray to assess Parkinson's disease models and the effects of neuroprotective drugs. Trends Pharmacol Sci 24:184-191

Tekumalla P. K. et al., 2001, Elevated levels of DeltaFosB and RGS9 in striatum in Parkinson's disease. Biol Psychiatry 50:813-816

Petroske et al., 2001, Mouse model of parkinsonism: A comparison. between subacute MPTP and chronic MPTP/probenecid treatment, Neuroscience 106(3): 589-601

Rajagopal et al., 1995, Epidermal growth factor expression in human colon and colon carcinomas: anti-sense epidermal growth factor receptor RNA down-regulates the proliferation of human colon cancer cells. Int. J. Cancer 62: 661-667

Dahiya et al., 1996, Differential gene expression of transforming growth factors alpha and beta, epidermal growth factor, keratinocyte growth factor, and their receptors in fetal and adult human prostatic tissues and cancer cell lines, Urology 48: 963-970

Zhang et al., 1999, Structure, alternative splicing, and expression of the human RGS9 gene. Gene 240, 23

Meshorer and Soreq, 2006, Virtues and woes of AChE alternative splicing in stress-related neuropathologies. Trends Neurosci 29, 216

Berke et al., 2001, Dopamine and glutamate induce distinct striatal splice forms of Ania-6, an RNA polymerase II-associated cyclin. Neuron 32, 277

Papa, 2002, The NDUFS4 nuclear gene of complex I of mitochondria and the cAMP cascade. Biochim. Biophys. Acia 1555, 147

Hirst et al, 2003, The nuclear encoded subunits of complex I from bovine heart mitochondria. Biochim. Biophys. Acta 1604, 135

Petruzzella, 2005, Mutations in the NDUFS4 gene of mitochondrial complex I alter stability of the splice variants. FEBS Letters 579, 3770

What is claimed is:

1. A method of discovering biomarkers indicative of an idiopathic neurodegenerative disease in a mammalian species, the method comprising:
(a) providing a test subject from the mammalian species, the test subject having been exposed to an environmental factor that causes a neurodegenerative disease or having been positively diagnosed with a neurodegenerative disease;
(b) obtaining RNA from a tissue of the test subject;
(c) determining the amounts of a first splice variant mRNA of a precursor-messenger RNA (pre-mRNA) of a gene of the test subject, a second splice variant mRNA of the same pre-mRNA of the same gene and total 18S RNA;
(d) determining for the test subject a first ratio of the amount of the first splice variant mRNA to the amount of the second splice variant mRNA or determining for the test subject a second ratio of the amount of the first splice variant mRNA to the amount of total 18S RNA;
(e) obtaining a third ratio of a control subject of the amount of the first splice variant mRNA to the amount of the second splice variant mRNA or a fourth ratio of a control subject of the amount of the first splice variant mRNA to the amount of total 18S RNA;
(f) comparing the first ratio to the third ratio to determine a first difference or comparing the second ratio to the fourth ratio to determine a second difference; and
(g) identifying the first splice variant mRNA as a biomarker indicative of the neurodegenerative disease for the mammalian species if the first difference or the second difference is not zero.

2. The method of claim 1 wherein the mammalian species is human.

3. The method of claim 1 wherein the neurodegenerative disease is Parkinson's disease.

4. The method of claim 1 wherein the neurodegenerative disease is Alzheimer's disease.

5. The method of claim 1 wherein the subject is unintentionally exposed to the environmental factor.

6. The method of claim 1 wherein the subject is intentionally exposed to the environmental factor.

7. The method of claim 1 wherein the change is an increase or a decrease.

8. The method of claim 1 wherein the exposure to the environmental factor is acute or chronic.

9. The method of claim 1 wherein the tissue is blood or cerebral spinal fluid (CSF).

10. A biomarker indicative of an idiopathic neurodegenerative disease in a subject of a mammalian species, the subject having a first amount of a first splice variant mRNA of a precursor-messenger RNA (pre-mRNA) transcript of a gene in the mammalian subject in a tissue from the subject, a second amount of a second splice variant mRNA of the same precursor-messenger RNA (pre-mRNA) transcript of the same gene in the same tissue, and an amount of total 18S RNA in the same tissue, and the biomarker for the mammalian species comprises the first splice variant mRNA wherein the first splice variant mRNA satisfies one of the conditions selected from the group consisting of:
(a) a ratio of the amount of the first splice variant mRNA to the amount of the second splice variant mRNA of the mammalian subject having been exposed to an environmental factor that causes the neurodegenerative disease or having been positively diagnosed with the neurodegenerative disease is different from a ratio of the amount of the first splice variant mRNA to the amount of the second splice variant mRNA in a control subject who does not have the neurodegenerative disease; and
(b) a ratio of the amount of the first splice variant mRNA to the amount of total 18S RNA of the mammalian subject having been exposed to an environmental factor that causes the neurodegenerative disease or having been positively diagnosed with the neurodegenerative disease is different from a ratio of the amount of the first splice variant mRNA to the amount of total 18S RNA in a control subject who does not have the neurodegenerative disease.

11. The splice variant mRNA of claim 10 wherein the mRNA is selected from the group consisting splice variant transcripts from the genes FosB, RGS9, Ania6, AChE and NDUFS4.

12. The splice variant mRNA of claim 10 wherein the mammalian species is human.

13. The splice variant mRNA of claim 10 wherein the tissue is blood or cerebral spinal fluid (CSF).
